(19) United States
(12) Patent Application Publication
(10) Pub. No.: US 2006/0228428 A1
(43) Pub. Date: Oct. 12, 2006

(54) A composition for preventing plant diseases resulted from infection of plant pathogens and a method for preparing the same

(76) Inventors: Hoon Serg Kang, Masan-si (KR); Sang Hoon Paik, Sungnam-si (KR); In Whan Jang, Pyungtaek-si (KR); Myung-Suk Choi, Jinju-si (KR); Jeom Hyun Baek, Changwon-si (KR); Jae Gon Kang, Jinhae-si (KR); Jun Seuk Lee, Soonsung-gu (KR); Jin Mee An, Haman-gun (KR); So-Young Kim, Jinju-si (KR); Sang Hoon Han, Masan-si (KR)

Correspondence Address: JHK LAW, P.O. BOX 1078, LA CANADA, CA, 91012-1078, US

(21) Appl. No.: 11/278,535

(22) Filed: Apr. 3, 2006

(30) Foreign Application Priority Data
Apr. 7, 2005 (KR) 10-2005-0028993

(51) Int. Cl. A61K 36/66; A61K 31/4741; A61K 36/185

(52) U.S. Cl. 424/725; 424/749; 514/283

(57) ABSTRACT

The present invention relates to a composition for preventing at least one plant disease resulted from infection of plant pathogens and a method for preparing the same. Particularly, the composition contains extract of at least one plant selected from the group consisting of Coptis chinensis Franch., Phellodendron genus plant, Sanguinaria canadensis and Chelidonium majus var. asiaticum which are natural plants having anti-plant pathogen activity or berberine which is purified therefrom. The composition of the present invention can be effectively used for preventing disease pathogens such as plant powdery mildew, anthracnose of pepper, damping-off of cucumber and gray mold rot of strawberry resulted from infection of plant pathogens such as Colletrotrichum gloeosporiodes, Pythium ultimum or Botrytis cinerea. For an alternative method for preparing the composition comprising berberine and/or derivatives thereof, the present invention provides various optimal culture condition and two phases cultivation methods using Phellodendron genus plant.

### RELATED APPLICATIONS

Priority of Korean Patent Application No. 2005-28993 filed on Apr. 7, 2005 is claimed. The co-pending application is incorporated by reference herein.

### TECHNICAL FIELD

The present invention relates to a composition for preventing at least one plant disease resulted from infection of plant pathogens containing extract of natural plants and a method for preparing the same.

### BACKGROUND

Powdery mildew is one of the most widespread and easily recognized plant diseases in the world. They affect virtually all kinds of plants such as cereals, grasses, vegetables, ornamentals, weeds, shrubs, fruit trees, broad-leaved shade and forest trees. In Korea, 336 plant species among 2,987 species can be infected with powdery mildew (Shin, 1994, Mycobiology, 22:355-365). An alternative control for powdery mildew is environmental control, selection of pest-resistant species, hygiene control, chemical treatment and using non-toxic compounds such as natural plant products. However, it has been reported that there are various organic pesticide-resistant pathogens and uses of chemical or organic pesticides have tended to decrease strongly. Although powdery mildew can be partially prevented by environmental control, reliable prevention is difficult because the pathological progress of the disease is affected by each environment. For example, although the symptoms of powdery mildew can be alleviated by low humidity, spraying in leaf surface and rapid drying, the symptoms of powdery mildew can become worse rapidly by change of humidity. Organic pesticides are very effective but they reduce useful insects and organic pesticide-resistant microorganism can be generated. In addition, organic pesticides result in some problem such as direct toxicity and remnants of toxic compounds in human and animal. Hence, the development of non-toxic natural pesticide is needed.

Coptis is a small, perennial herb found in North Korea. It is also known by a variety of names, including goldthread and yellow root, because the plant's root, is golden yellow in color. In traditional Chinese medicine, it is used for gastrointestinal problems, diarrhea, hypertension and bacterial infections. The plant's roots contain berberine, which can be used as anti-inflammatory and antibacterial, and berberine-like alkaloids which are believed to facilitate healing.

For thousands of years, the bark from the Phellodendron tree (Amur Cork tree) has been used as an ingredient in herbal formulas in Chinese medicine. Phellodendron genus plant has been used to treat gastric ulcers, bacterial infections, fungal infections and diabetes. It has also been used for immuno-suppression and as a topical anti-inflammatory agent. Particularly, Phellodendron amurense and Phellodendron insulare belong to Phellodendron genus.

Sanguinaria canadensis (bloodroot) is grown in the mountains of America. It is most persistent and possibly valid use takes advantage of the flesh destroying properties of the root juice or powered root for treating conditions of the skin such as ringworm, warts, polyps, fungal growths, anticancer, and the like. Extract of Sanguinaria canadensis has long been used in toothpaste and mouthwash to fight plaque and gingivitis and use thereof is now sanctioned by the U.S. Food and Drug Administration. The root of Sanguinaria canadensis has been used internally, in very small doses, to stimulate the digestive system and as an emetic.

Celandine (Chelidonium majus var. asiaticum) is a biennial or perennial plant widespread in damp, rich soil in the north-eastern U.S. and along fences, roads, hedges, as well as in waste places, in Europe. Taken internally, celandine has a special effect on the digestive system (stomach, gallbladder, liver), and its antispasmodic properties make it useful for asthmatic symptoms. The juice of celandine has some antiseptic properties and has long been used to remove warts. The juice of celandine is mixed with vinegar when using it on the skin.

In these herbal plants, anti-plant pathogenic effects against plant fungi pathogens especially, powdery mildew has been not yet reported. The present inventors determined whether the extract of the above herbal plants have anti-plant pathogenic activity against various plant pathogens and elucidated what is an effective compound for the anti-plant pathogenic activity from the extracts.

In the meantime, active compounds of plants having certain pharmaceutical and/or agricultural applicability can be supplied by direct extraction of useful plant resources. However, the above-mentioned method for preparing the active compounds is problematic due to the restriction of cultivated regions and fluctuation in market price according to unstable supply. Thus, biotechnological methods such as cell cultures are needed. Although in the past years all economically important plants have been brought into cell culture, however, in most cases the productivity was too low to allow an economically feasible process. Selection of stable cell line and finding of optimum conditions for cell growth can solve these problems. Optimal conditions for the increase of both cell growth and product yield can be found by changing the chemical and physical factors in culture.

Through intensive and laborious works, the present inventors perfected the present invention by confirming that the extract could prevent growth of various plant pathogens effectively and the active compound is berberine. Particularly, the extract of the present invention, berberine and derivatives thereof showed anti-pathogenic activities against anthracnose of pepper, damping-off of cucumber, gray mold rot of strawberry. In addition, the present inventors established various optimal culture condition of Phellodendron insulare for the alternative method for preparing the active compound, berberine.

### BRIEF DESCRIPTION OF THE DRAWINGS

FIG. 1 is a schematic diagram showing extraction procedure I of anti plant pathogen composition from Coptis chinensis Franch., Phellodendron genus plant, Sanguinaria canadensis or Chelidonium majus var. asiaticum.

FIG. 2 is a schematic diagram showing extraction procedure II of anti-plant pathogen composition from Coptis chinensis Franch., Phellodendron genus plant, Sanguinaria canadensis or Chelidonium majus var. asiaticum.

FIG. 3 is a photograph of thin layer chromatography (TLC) illustrating the extracts of 4 plants and authentic standard berberine:

Lane 1: Coptis chinensis Franch.;

Lane 2: Phellodendron amurense;

Lane 3: Sanguinaria canadensis;

Lane 4: Chelidonium majus var. asiaticum; and

Lane 5: berberine.

FIG. 4 is a structure of berberine and derivertives thereof.

FIG. 5a is a chromatogram showing a result of HPLC (high performance liquid chromatography) of upper (berberine) spot of TLC separation; and

FIG. 5b is a chromatogram showing a result of HPLC of lower spot of TLC separation.

FIG. 6a is a ¹H NMR (nuclear magnetic resonance) spectrum of berberine-Cl; and

FIG. 6b is a ¹H NMR (nuclear magnetic resonance) spectrum of Coptis Rhizoma.

### SUMMARY OF THE INVENTION

An object of the present invention is to provide a composition for preventing at least one plant disease resulted from infection of a plant pathogen containing extract of natural plants or an active compound therefrom and a method for preparing the same.

Another object of the present invention is to provide a method for preparing the active compound and derivatives thereof by culturing the plant.

Further object of the present invention is to provide a method for preventing at least one plant disease resulted from infection of a plant pathogen.

In order to achieve the aforementioned objects, the present invention provides a composition for preventing at least one plant disease resulted from infection of a plant pathogen containing extract of at least one plant selected from the group consisting of Coptis chinensis Franch., Phellodendron genus plant, Sanguinaria canadensis and Chelidonium majus var. asiaticum by organic solvent and/or water or purified berberine or derivatives thereof, or synthesized berberine or derivatives thereof.

The present invention also provides a method for preparing the composition for preventing at least one plant disease resulted from a plant pathogen comprising the following steps:

1) extracting at least one plant selected from the group consisting of Coptis Rhizoma (Coptis chinensis Franch.), Phellodendron genus plant, Sanguinaria canadensis and Chelidonium majus var. asiaticum by water, methanol or ethanol or combination thereof after cutting;

2) sedimenting and filtering of extract of step 1; and

3) concentrating extract of step 2.

The present invention also provides a method for preparing berberine or derivatives thereof comprising the following steps:

1) culturing callus of Phellodendron genus plant using a growth medium (pH 5.8) comprising 0.048˜0.19% of KNO₃, 0.009˜0.036% of MgSO₄H₂O, 0.00085˜0.0034% of MnSO₄7H₂O, 0.00043˜0.0017% of ZnSO₄7H₂O, 0.00000013˜0.000005% of CuSO₄5H₂O, 0.017˜0.066% of CaCl₂2H₂O, 0.000042˜0.00017% of KI, 0.00000013˜0.000005% of CoCl₂2H₂O, 0.083˜0.33% of NH₄NO₃, 0.00031˜0.0012% of H₂BO₃, 0.00000013˜0.000005% of Na₂MoO₄2H₂O, 0.0043˜0.017% of NH₂PO₄, 0.0018˜0.0073% of NaFeEDTA, 0.0005˜0.002% of indole-3-acetic acid (IAA), 0.000025˜0.0001% of benzylamino purine (BA) and 5˜9% of sucrose;

2) culturing the callus of step 1 using a production medium comprising 0.095˜0.38% of KNO₃, 0.0125˜0.05% of MgSO₄H₂O, 0.00085˜0.0034% of MnSO₄7H₂O, 0.00043˜0.0017% of ZnSO₄7H₂O, 0.00000013˜0.000005% of CuSO₄5H₂O, 0.017˜0.066% of CaCl₂2H₂O, 0.000042˜0.00017% of KI, 0.00000013˜0.000005% of CoCl₂2H₂O, 0.083˜0.33% of NH₄NO₃, 0.00015˜0.0006% of H₂BO₃, 0.00000013˜0.000005% of Na₂MoO₄2H₂O, 0.017˜0.068% of NH₂PO₄, 0.0014˜0.0056% of NaFeEDTA, 0.0005˜0.002% of indole-3-acetic acid (IAA) and 5˜9% of sucrose; and

3) purifying berberine or derivatives thereof from the cultured callus of step 2.

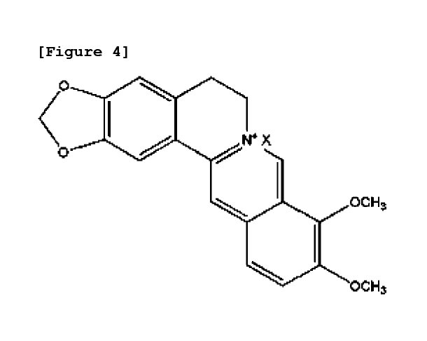

In the step 3, samples for berberine analysis were collected by taking 0.5 g of cells (fresh weight) and analyzed using the described method by Nakagawa (1984). Dried cells were then extracted with 10 ml methanol in a sonicating bath at 50° C. for 1 hour. Quantitative determinations of berberine and palmatine were made by the calibration curve obtained with authentic compounds using a Gilson HPLC system equipped with a UV detector. Separation was accomplished using p-Bondapak C18 (300×3.9 mm) column; solvent 1 mM tetrabutylammonium phosphate (pH 2.0): acetonitrile (40:60); flow rate 0.5 ml/min; wavelength 265 nm. Berberine was tentatively identified based on retention time and UV spectra (FIG. 4).

The present invention provides a method for preventing at least one plant disease resulted from infection of a plant pathogen comprising treating said composition to plants.

Detailed description of the present invention are below:

### DETAILED DESCRIPTION OF THE INVENTION

The present invention provides a composition for preventing at least one plant disease resulted from a plant pathogen containing extract of at least one plant selected from the group consisting of Coptis chinensis Franch., Phellodendron genus plant, Sanguinaria canadensis and Chelidonium majus var. asiaticum by organic solvent and/or water or purified berberine or derivatives thereof, or synthesized berberine or derivatives thereof.

In an embodiment of the present invention, the Phellodendron genus plant is Phellodendron amurense or Phellodendron insulare but not limited thereto.

In another embodiment of the present invention, the plant disease is plant powdery mildew, damping-off of cucumber or gray mold rot of strawberry but not limited thereto.

In another embidiment of the present invention, the plant pathogen is Colletrotrichum gloeosporiodes, Pythium ultimum or Botrytis cinerea but not limited thereto.

The inventors of the present invention determined that the composition containing extract of Coptis chinensis Franch., Phellodendron genus plant, Sanguinaria canadensis or Chelidonium majus var. asiaticum has anti-pathogenic activities against anthracnose of pepper, damping-off of cucumber. In this regard, the crude extracts showed strong anti-plant pathogenic activities against anthracnose of pepper, damping-off of cucumber, gray mold rot of strawberry (Table 1). The inventors of the present invention also determined anti-plant pathogenic activities of the extract against powdery mildew of strawberry. The extract lowered contraction rate of powdery mildew when it was treated at various concentration. The extract had anti-pathogenic activities against Colletrotrichum gloeosporiodes, Pythium ultimum and Botrytis cinerea which are pathogens of anthracnose of pepper, damping-off of cucumber, gray mold rot of strawberry, respectively. In a preferred embodiment, the Phellodendron genus plant is Phellodendron amurense or Phellodendron insulare but not limited thereto.

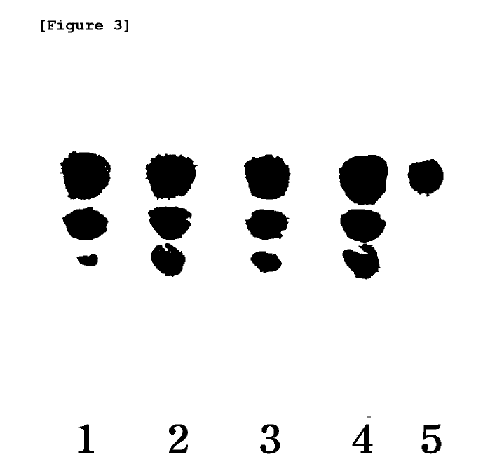

The inventors of the present invention analyzed thin layer chromatogram of the extract and standard berberine and collected a band corresponding to the standard berberine and then purified berberine compound by high performance liquid chromatography (HPLC) (FIG. 3). Berberine fraction separated by TLC and HPLC also showed strong anti-pathogenic activities against the aforementioned pathogens. A clear zone is generated around a paper disk treated with this fraction. This result indicates that the extract prepared by the method of the present invention showed strong antifungal activity. The active compound existing in the extract was determined as berberine and derivatives. Concentration of berberine in the extract is 10 μg/ml to 1000 μg/ml (Table 8).

The active antifungal composition can be directly used as anti-pathogenic agents in the cereals (corn, wheat, rice and sugarcane), cotton, tobacco, vegetables (cucumber, red pepper, lettuce, anion, tomato and black pepper etc.), strawberry, potato, soybean, tea tree, coffee tree, banana, walnut, ornamental trees, grasses and forest etc.

The present invention also provides a method for preparing said composition for preventing at least one plant disease resulted from infection of a plant pathogen comprising steps of:

1) extracting at least one plant selected from the group consisting of Coptis Rhizoma (Coptis chinensis Franch.), Phellodendron genus plant, Sanguinaria canadensis and Chelidonium majus var. asiaticum by water, methanol or ethanol or combination thereof after cutting;

2) sedimenting and filtering of extract of step 1; and

3) concentrating extract of step 2.

In an embodiment of the present invention, the Phellodendron genus plant is Phellodendron amurense or Phellodendron insulare but not limited thereto.

In another embodiment of the present invention, the plant disease is plant powdery mildew, damping-off of cucumber or gray mold rot of strawberry but not limited thereto.

In another embodiment of the present invention, the plant pathogen is Colletrotrichum gloeosporiodes, Pythium ultimum or Botrytis cinerea but not limited thereto.

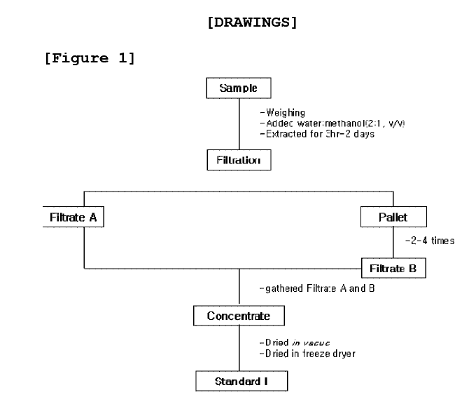

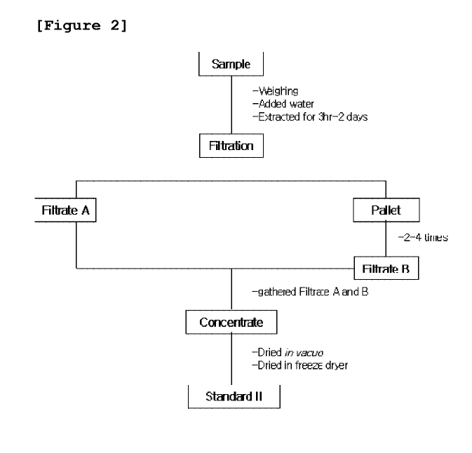

For obtaining the above extract, Coptis Rhizoma, Phellodendron amurense, Sanguinaria canadensis or Chelidonium majus var. asiaticum may be collected in the field and mountain or purchased in a market, dried in the shade, and then extracted by water or organic solvents (FIGS. 1 and 2). The extract can be obtained in the forms of liquid and/or powder, and if not mentioned particularly herein, it is powder.

The present invention further provides a method for preparing berberine or derivatives thereof comprising the following steps:

1) culturing a callus of Phellodendron genus plant using a growth medium;

2) culturing the callus of step 1 using a production medium; and

3) purifying berberine or derivatives thereof from the cultured callus of step 2.

In a preferred embodiment of the method for preparing berberine or derivatives thereof, the growth medium comprises 0.048˜0.19% of KNO₃, 0.009˜0.036% of MgSO₄H₂O, 0.00085˜0.0034% of MnSO₄7H₂O, 0.00043˜0.0017% of ZnSO₄7H₂O, 0.00000013˜0.000005% of CuSO₄5H₂O, 0.017˜0.066% of CaCl₂2H₂O, 0.000042˜0.00017% of KI, 0.00000013˜0.000005% of CoCl₂2H₂O, 0.083˜0.33% of NH₄NO₃, 0.00031˜0.0012% of H₂BO₃, 0.00000013˜0.000005% of Na₂MoO₄2H₂O, 0.0043˜0.017% of NH₂PO₄, 0.0018˜0.0073% of NaFeEDTA, 0.0005˜0.002% of indole-3-acetic acid (IAA) but not limited thereto.

In another preferred embodiment of the method for preparing berberine or derivatives thereof, the production medium comprises 0.095˜0.38% of KNO₃, 0.0125˜0.05% of MgSO₄H₂O, 0.00085˜0.0034% of MnSO₄7H₂O, 0.00043˜0.0017% of ZnSO₄7H₂O, 0.00000013˜0.000005% of CuSO₄5H₂O, 0.017˜0.066% of CaCl₂2H₂O, 0.000042˜0.00017% of KI, 0.00000013˜0.000005% of CoCl₂2H₂O, 0.083˜0.33% of NH₄NO₃, 0.00015˜0.0006% of H₂BO₃, 0.00000013˜0.000005% of Na₂MoO₄2H₂O, 0.017˜0.068% of NH₂PO₄, 0.0014˜0.0056% of NaFeEDTA, 0.0005˜0.002% of indole-3-acetic acid (IAA) and 5˜9% of sucrose but not limited thereto. The percentage of each nutrient is weight per volume (w/v), if not mentioned specifically.

In another preferred embodiment of the method for preparing berberine or derivatives thereof, the Phellodendron genus plant is Phellodendron amureus or Phellodendron insulare but not limited thereto. In a more preferred embodiment of the method of the present invention, the Phellodendron genus plant is Phellodendron insulare.

The present invention provides also a method for preventing at least one plant disease resulted from infection of a plant pathogen comprising treating said composition to plants.

In a preferred embodiment of the present invention, the plant disease is plant powdery mildew, damping-off of cucumber or gray mold rot of strawberry but not limited thereto.

In another preferred embodiment of the present invention, the plant pathogen is Colletrotrichum gloeosporiodes, Pythium ultimum or Botrytis cinerea but not limited thereto.

Practical and presently preferred embodiments of the present invention are illustrated as shown in the following Examples.

However, it will be appreciated that those skilled in the art, on consideration of this disclosure, may make modifications and improvements within the spirit and scope of the present invention.

### EXAMPLE 1

### Preparation of Anti-Pathogen Composition

Collected or purchased plant materials including Coptis Rhizoma (Coptis chinensis Franch.), Phellodendron amurense, Sanguinaria canadensis and Chelidonium majus var. asiaticum was dried in the shade, and then macerated with 20 mesh size. 100 g of the macerated plant materials were extracted with 500 ml distilled water or solvent consisting of water and ethanol (or methanol) with ratio of 2:1 in the extractor at room temperature for 24 hours and filtered. Crude extract was vacuum dried, and dried using freeze dryer (FIGS. 1 and 2). The freeze dried powder was defined as “standard extract”.

### EXAMPLE 2

### Antifungal Assay of the Anti-Pathogen Composition

For assessing antifungal activity of the standard extract prepared by Example 1, we performed antifungal assay against Colletrotrichum gloeosporiodes, Pythium ultimum, and Botrytis cinerea using the standard extract as follows: Crude extract was diluted serially to 1.6, 0.8, 0.4, 0.2 and 0.1 mg/l, and filter sterilized with membrane filter (0.2 μm, Gelman). We added 2 ml sterilized extracts to 18 ml of sterilized potato dextrose agar (PDA) medium and then let the PDA medium cool and harden. Plant pathogens described above were cultured in medium at 29° C. for 5 days. A small piece of fungal mass (5 mm of diameter) of the pathogen was inoculated onto PDA medium and incubated at 29° C., for 4 days and then hyphal growth was measured. Through the assay relative growth inhibition rates (% GIR) was determined. The relative growth inhibition rate was calculated as follows:
100×{1−hyphal diameter of the standard extract-treated group/hypha diameter of non-treated group control)}

Further EC₅₀ which stands for effective concentrartion 50% was calculated using the growth inhibition rate curve. EC₅₀ values of anthracnose of pepper, damping-off of cucumber and gray mold rot of strawberry were 400 to 800 μg/ml (Table 1).

TABLE 1
Antifungal activity against some test
microorarnisms of plant extracts

| | | | | Gray mold |
|---|---|---|---|---|
| | Conc. | anthracnose | Damping-off | rot of |
| Extracts | (μg/ml) | of pepper | of cucumber | strawberry |
| Coptis | 100 | 51.3^{a)} | 65.3 | 31.3 |
| | 200 | 47.7 | 52.3 | 25.3 |
| | 400 | 41.3 | 46.7 | 19.7 |
| | 800 | 32.0 | 31.3 | 15.3 |
| | 1,600 | 26.0 | 22.3 | 11.3 |
| | Control | 60.3 | 78.7 | 36.3 |
| Phellodendron | 100 | 59.7 | 77.3 | 35.0 |
| | 200 | 55.3 | 70.3 | 31.7 |
| | 400 | 49.3 | 51.3 | 27.3 |
| | 800 | 38.3 | 43.3 | 18.3 |
| | 1,600 | 30.3 | 38.7 | 12.7 |
| | Control | 61.7 | 79.7 | 36.3 |
| Sanguinaria | 100 | 58.7 | 75.3 | 32.3 |
| | 200 | 56.3 | 69.7 | 24.7 |
| | 400 | 46.3 | 52.3 | 19.7 |
| | 800 | 39.7 | 38.3 | 15.3 |
| | 1,600 | 31.0 | 29.7 | 12.0 |
| | Control | 59.7 | 77.0 | 34.7 |
| Chelidonium | 100 | 55.7 | 67.3 | 30.3 |
| majus var. | 200 | 42.3 | 52.3 | 23.3 |
| asiaticum | 400 | 37.3 | 39.7 | 17.3 |
| | 800 | 29.7 | 31.3 | 11.7 |
| | 1,600 | 22.7 | 25.3 | 9.7 |
| | Control | 61.3 | 72.7 | 33.3 |

^{a)}hypal growth (mm)

### EXAMPLE 3

### Isolation and Elucidation of Active Antifungal Compounds from Extract of 4 Plant Species and Analysis of Antimicrobial Activity of Derberine and Derivtives Thereof

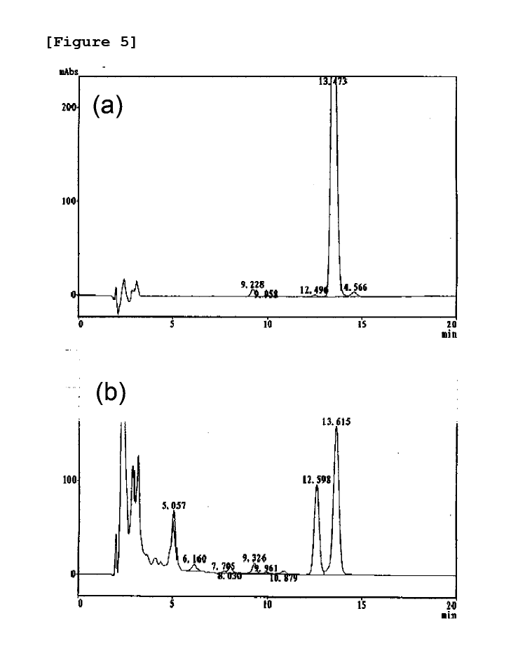

The standard extract prepared by Example 1 and a berberine standard (Sigma, USA) were ran on TLC plate in solvent consising of chlroroform, methanol and H₂O with the ratio of 5:1:0.1 and bands were detected by UV lamp. The TLC analysis was performed according to a known method. Among various bands of the standard extract, one corresponding to berberine standard and the other spot was separated by HPLC (FIG. 5). The HPLC condition indicated Table 2.

TABLE 2
Condition of HPLC analysis

| Parameter | Condition |
|---|---|
| Column | μBondpak C₁₈Column(4.6 mm × 250 mm) |
| Solvent system | 0.1% H₃PO₄ 50%, CH₃CN 50%, SDS 0.6% |
| Flow rate | 1.5 ml/min |
| Detector | UV visible 254 mm |
| Injection volume | Volume 10 μL |

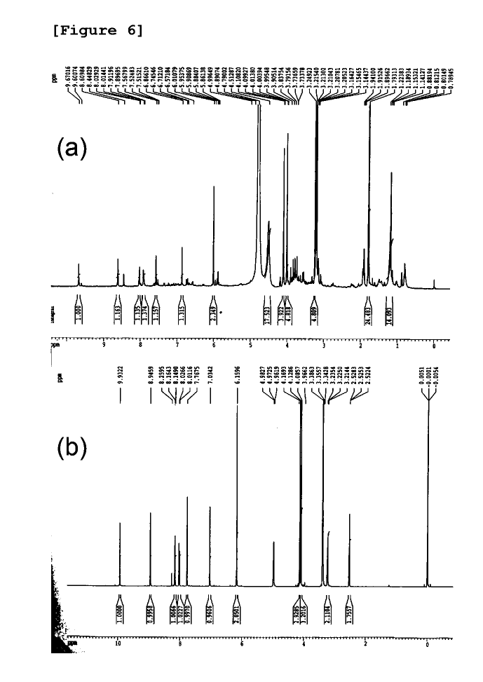

HPLC analysis was performed by HPLC grade solvent and distilled water which was demineralized after by 0.45 μm filter. Other reagents for extracting and preparation were first grade. 0.5 mg of berberine standard (Sigma, USA) was dissolved in 5 ml methanol and then a calibration curve was drawn after injection of 5, 10, and 50 μL, respectively. As a result of the HPLC analysis, a peak whose retention time is same with berberine standard was formed in the standard extract. Further, we investigated berberine standard and compounds derived form Chelidonium majus var. asiaticum using ¹H NMR (nuclear magnetic resonance) analysis (FIG. 6). As shown in FIG. 6, the appearance of peaks was similar.

To confirm antimicrobial activity of berberine and derivative thereof, we collected TLC bands of berberine and derivative thereof and investigated antimicrobial activity of berberine and derivative thereof against known plant pathogens using paper disk method (Ericsson, H., Hogman, C & Wickman. K. 1954, Scandinavian Journal of Clinical Laboratory Investigation 6, Suppl. 11:21-26). In result, as shown in Table 6, we confirmed that an inhibitory zone was clearly generated around a paper disk treated with berberine and derivatives thereof. Therefore, the result of the aforementioned HPLC analysis, ¹H NMR analysis and antimicrobial activity, the active antimicrobial compound exisisting in the extract of Coptis Rhizoma, Phellodendron genus plant, Sanguniaria canadensis, and Chelidonuum majus var. asiaticum was identified as berberine or derivatives thereof.

TABLE 3
Growth inhibition of berberine and its derivatives
by purified from TLC against plant pathogens.

| | | | gray mold |
|---|---|---|---|
| | glomellera | damping-off | rot of |
| treatments | cingurata | of cucumber | strawberry |
| Captis Rhizoma | ++^{a)} | ++ | + |
| Phellodendron | + | + | + |
| amurense | | | |
| Sanguinaria | + | + | + |
| canadensis | | | |
| Chelidonium majus | + | + | + |
| var. asiaticum | | | |
| berberine chloride | ++ | ++ | + |
| berberine iodide | + | + | + |
| berberine bromine | + | + | + |
| Control | − | − | − |

^{a)}++: strong inhibition,
+: moderate inhibition,
−: no inhibition.

### EXAMPLE 4

### Prevention Effect Against Powdery Mildew of Each Extract and Berberine and Derivatives Thereof

To measure prevention effect against powdery mildew of extract of Coptis Rhizoma, Phellodendron amurense, Sanguniaria canadensis and Chelidonuum majus var. asiaticum and berberine and its derivative, we performed a preventive test agaist strawberry powdery mildew resulted from infection of Sphaerotheca aphanis var. aphanis using the standard extract obtained by Example 1 or berberine and derivative thereof. The above extract (30 mL) obtained from 10 g of fresh plant materials was dissolved in 200 μg/ml of Tween 20 solution. Final concentration of berberine and derivatives thereof was 400 μg/ml. Negative control was prepared by adding 250 μg/ml of Tween 20 in distilled water. As a positive control reagent, 30% solution of Rifzol wettable powder (Rifzol WP, Aventis Corp Science, Korea) is used and the Rifzol solution was diluted 1:4000 and treated with amount of 75 μg/ml which is common packing concentration.

In house condition of sandy-loamy soil, disease incidence rate was determined 7 days after treatment of the above mentioned reagents compared to one of non-treatment control and the test was repeated 3 times by randomized Block Design.

Before treatment of the reagents, the disease incidence rate of strawberry powdery mildew was 34.1%. The disease incidence rate is defined as percentage of disease developed plantsamong total investigated plants. In result, as shown Table 4, Coptis Rhizoma, Phellodendron genus plant, Sanguniaria canadensis, and Chelidonuum majus var. asiaticum and berberine and derivatives thereof showed excellent effect on prevention of strawberry mildew.

TABLE 4
Disease incidence rate and prevention effects against strawberry powdery mildew
7 days after treatment of plant extract

| | | DIR B²⁾ (%) | | | | | |
|---|---|---|---|---|---|---|---|
| Treatments | DIR A¹⁾ (%) | I | II | III | MV³⁾ | PR⁴⁾ (%) | SD⁵⁾ |
| Coptis | 34.1 | 4.3 | 4.2 | 6.0 | 4.8 | 74.7 | ab |
| Rhizoma | | | | | | | |
| Phellodendron | 32.4 | 7.1 | 10.3 | 11.4 | 9.6 | 49.5 | c |
| amurense | | | | | | | |
| Sanguniaria | 32.2 | 12.0 | 6.9 | 6.1 | 8.3 | 56.3 | bc |
| canadensis | | | | | | | |
| Chelidonuum | 30.8 | 5.4 | 4.9 | 4.9 | 5.1 | 73.2 | ab |
| majus var. | | | | | | | |
| asiaticum | | | | | | | |
| berberine | 29.5 | 5.9 | 5.1 | 5.1 | 5.8 | 69.5^{c} | ab |
| chloride | | | | | | | |
| berberine | 33.3 | 7.1 | 6.6 | 6.6 | 7.0 | 63.2 | abc |
| iodide | | | | | | | |
| berberine | 32.1 | 5.2 | 6.1 | 6.1 | 6.3 | 66.8 | abc |
| bromine | | | | | | | |
| positive | 28.1 | 4.6 | 2.7 | 2.7 | 3.7 | 80.5 | a |
| Control | | | | | | | |
| (Rifzol WP) | | | | | | | |
| negative | 34.1 | 13.8 | 23.1 | 23.1 | 19.0 | — | |
| control | | | | | | | |

¹⁾DIR A: disease incident rate before treatment
²⁾DIR B: disease incident rate 7 days after treatment
³⁾MV: mean value
⁴⁾PR: prevention rate
⁵⁾SD: significant difference according to DMRT (Duncan's multiple range test)

### EXAMPLE 5

### Prevention Effect Against Powdery Mildew at Various Concentration of Extracts of 4 Species and Berberine and Derivative Thereof

We investigated preventive effects of standard forms of berberine chloride, berberine iodide and berberine bromide and the standard extract obtained by Example 1 against strawberry powdery mildew at various concentrations according to the method of Example 4. In result, as shown in Table 5, each extract showed prominent preventive effects at all concentrations and such effects are similar with that of common packing concentration (75 μg/ml) of Rifzol, a positive control. Therefore, the standard extract of the present invention, berberine and derivatives thereof showed preventive effects on strawberry powdery mildew depending on concentration. When the freeze-dried standard extract obtained by Example 1 was used at concentration of at least 1 μg/ml or preferably 10 μg/ml, it prevented strawberry powdery mildew resulted from an infection of Sphaerotheca aphanis var. aphanis. From the above effect, the present inventors confirmed that the standard extract could substitute for synthetic chemical pesticides.

TABLE 5
Disease incidence rate and preventive effecs against strawberry powdery mildew based
according to various concentrations

| extract or | | Conc. | | DIR B²⁾ (%) | | | | | |
|---|---|---|---|---|---|---|---|---|---|
| compounds | treatments | (μg/ml) | DIR A¹⁾ | I | II | III | MV³⁾ | PR⁴⁾ (%) | SD⁵⁾ |
| Coptis | | 1000 | 34.3 | 4.3 | 4.2 | 8.5 | 5.7 | 83.9 | a |
| | | 300 | 39.1 | 8.2 | 6.9 | 9.2 | 8.1 | 77.1 | ab |
| | Extract | 100 | 32.5 | 10.3 | 12.1 | 8.9 | 10.4 | 70.5 | b |
| | | 10 | 33.1 | 25.3 | 22.1 | 19.7 | 22.4 | 36.5 | c |
| | | 1 | 33.6 | 30.1 | 29.3 | 27.8 | 29.1 | 17.6 | d |
| | Rifzol | 75 | 35.3 | 6.9 | 5.2 | 2.3 | 4.8 | 86.4 | a |
| | Control | — | 33.1 | 32.4 | 38.9 | 34.7 | 35.3 | — | — |
| Phellodendron | | 1000 | 33.1 | 10.5 | 8.2 | 9.3 | 9.3 | 73.7 | b |
| | | 300 | 35.4 | 16.2 | 17.8 | 15.8 | 16.6 | 53.0 | c |
| | Extract | 100 | 36.5 | 18.4 | 19.3 | 14.7 | 17.5 | 50.4 | c |
| | | 10 | 35.4 | 24.3 | 22.8 | 25.9 | 24.3 | 31.2 | d |
| | | 1 | 32.9 | 31.2 | 32.7 | 32.1 | 32.0 | 9.3 | E |
| | Rifzol | 75 | 35.3 | 6.9 | 7.2 | 2.3 | 5.5 | 84.4 | a |
| | Control | — | 33.1 | 32.4 | 38.9 | 34.7 | 35.3 | — | — |
| Sanguniaria | | 1000 | 36.7 | 11.2 | 8.4 | 6.2 | 8.6 | 75.6 | ab |
| | | 300 | 37.1 | 14.3 | 12.4 | 11.1 | 12.6 | 64.3 | bc |
| | Extract | 100 | 29.9 | 13.7 | 15.4 | 18.3 | 15.8 | 55.2 | c |
| | | 10 | 30.2 | 23.1 | 25.4 | 28.7 | 25.7 | 27.2 | d |
| | | 1 | 33.4 | 29.7 | 28.5 | 32.1 | 30.1 | 14.7 | d |
| | Rifzol | 75 | 35.3 | 6.9 | 7.2 | 2.3 | 5.5 | 84.4 | a |
| | Control | — | 33.1 | 32.4 | 38.9 | 34.7 | 35.3 | — | — |
| Chelidonuum | Extract | 1000 | 39.1 | 10.2 | 7.4 | 6.9 | 8.2 | 76.8 | a |
| | | 300 | 35.7 | 11.5 | 14.3 | 17.3 | 14.4 | 59.2 | b |
| | | 100 | 38.7 | 15.1 | 16.3 | 18.4 | 16.6 | 53.0 | b |
| | | 10 | 37.0 | 21.1 | 23.4 | 25.6 | 23.4 | 33.7 | c |
| | | 1 | 39.1 | 35.4 | 33.7 | 41.2 | 27.8 | 21.2 | d |
| | Rifzol | 75 | 35.3 | 6.9 | 7.2 | 2.3 | 5.5 | 84.4 | a |
| | Control | — | 33.1 | 32.4 | 38.9 | 34.7 | 35.3 | — | — |
| Berberine | | 1000 | 33.5 | 3.4 | 8.7 | 4.2 | 5.4 | 84.7 | a |
| chloride | | 300 | 32.8 | 5.2 | 8.5 | 10.3 | 8.0 | 77.3 | a |
| | Extract | 100 | 34.7 | 8.2 | 7.7 | 12.1 | 9.3 | 73.7 | a |
| | | 10 | 30.1 | 23.4 | 22.1 | 19.9 | 21.8 | 38.2 | b |
| | | 1 | 32.2 | 30.2 | 30.8 | 32.1 | 31.0 | 12.2 | c |
| | Rifzol | 75 | 35.3 | 6.9 | 7.2 | 2.3 | 5.5 | 84.4 | a |
| | Control | — | 33.1 | 32.4 | 38.9 | 34.7 | 35.3 | — | — |
| berberine | | 1000 | 35.8 | 4.3 | 9.3 | 5.2 | 6.3 | 82.2 | a |
| iodide | | 300 | 32.9 | 6.2 | 7.9 | 8.4 | 7.5 | 78.8 | a |
| | Extract | 100 | 36.5 | 10.2 | 8.9 | 9.3 | 9.5 | 73.1 | a |
| | | 10 | 33.4 | 25.8 | 23.1 | 19.6 | 22.8 | 35.4 | b |
| | | 1 | 35.6 | 30.2 | 29.5 | 35.8 | 31.8 | 9.9 | c |
| | Rifzol | 75 | 35.3 | 6.9 | 7.2 | 2.3 | 5.5 | 84.4 | a |
| | Control | — | 33.1 | 32.4 | 38.9 | 34.7 | 35.3 | — | — |
| berberine | | 1000 | 32.5 | 5.9 | 9.4 | 7.2 | 7.5 | 78.8 | ab |
| bromine | | 300 | 34.3 | 8.9 | 12.9 | 8.3 | 10.0 | 71.7 | ab |
| | Extract | 100 | 33.6 | 13.2 | 11.7 | 15.8 | 13.6 | 61.5 | c |
| | | 10 | 35.4 | 13.5 | 17.6 | 24.8 | 18.6 | 47.3 | c |
| | | 1 | 33.1 | 29.6 | 27.6 | 35.4 | 30.9 | 12.5 | d |
| | Rifzol | 75 | 35.3 | 6.9 | 7.2 | 2.3 | 5.5 | 84.4 | a |
| | Control | — | 33.1 | 32.4 | 38.9 | 34.7 | 35.3 | — | — |

¹⁾DIR A: disease incident rate before treatment
²⁾DIR B: disease incident rate 7 days after treatment
³⁾MV: mean value
⁴⁾PR: prevention rate
⁵⁾SD: significant difference according to DMRT (Duncan's multiple range test)

### EXAMPLE 6

### Production of Berberine and Derivatives Thereof by Cell Culture

Primary callus cultures were performed from stem cambial tissues of cork tree (Phellodendron insulare) cultured on LS (Linsmier and Skoog, 1965, Physiol. Plant 18:100-127) medium supplemented with 0.5 mg/l 2,4-dichlorophenoxy acid (2,4-D), 0.1 mg/l benzylamino purine (BA), and 3% sucrose. Suspension culture of cork tree was initiated by transferring the callus to liquid MS medium supplemented with 3% sucrose, 1.0 mg/l 2,4-D, and 0.1 mg/l (BA). The suspension culture was placed in the same medium in the dark condition at 25° C. and was subcultured every 4 weeks. The effect of various macronutrients was tested by transferring duplicate samples of 1.5 g callus of 10-day old suspension culture into 250 ml Erlenmeyer flask containing 50 ml of test medium. The test media for analyzing the effect of nitrate, phosphate, sucrose, and growth regulators (2,4-D, IAA and BAP) were prepared by modifying concentration of each nutrient in the LS basal medium. All cultures were maintained under dark condition at 25° C. on a gyratory shaker. Samples for berberine analysis were collected by taking 0.5 g of cells (fresh weight) and analyzed using the method described by Nakagawa (1984, Plant Cell Rep 3:254-257). The effect of nitrogen source on both cell growth and berberine production was examined by varying concentration of nitrate and phosphate (Table 6). The highest berberine production was obtained in a medium containing 3550.0 mg/l of ammonium nitrate, while the highest cell growth was obtained in a medium containing 2600.0 mg/l of nitrate. Higher nitrate level than the above did not promote cell growth. However, cell growth was promoted exceptionally at the level of 85.0 mg/l phosphate (Table 6). The optimal concentration of phosphate for berberine production was 340.0 mg/l. The effect of sucrose on cell growth and berberine yield was investigated. Cell growth as well as berberine yield increased with increment of sucrose concentration (Table 6). The optimal concentration of sucrose for both berberine production and cell growth was 7%. In cork tree cells, cell growth and berberine production needed different growth regulators and IAA promoted growth of cells (Table 6). Although cell growth in mediums supplemented with high concentrations of 2,4-D and IAA markedly was superior to those supplemented with low concentrations or no growth regulator, berberine production was inhibited by high level of auxin.

TABLE 6
Composition of growth and production medium on
Phellodendron insulare cell cultures

concentration (mg/l)

| | | |
|---|---|---|
| | | production |
| components | growth medium | medium |

| | | | |
|---|---|---|---|
| macro and | KNO₃ | 950.000 | 1900.000 |
| micro | MgSO₄7H₂O | 180.540 | 250.000 |
| nutrients | MnSO₄H₂O | 16.900 | 16.900 |
| | ZnSO₄7H₂O | 8.600 | 8.600 |
| | CuSO₄5H₂O | 0.025 | 0.025 |
| | CaCl₂2H₂O | 332.020 | 332.020 |
| | KI | 0.830 | 0.830 |
| | CoCl₂6H₂O | 0.025 | 0.025 |
| | NH₄NO₃ | 1650.000 | 1650.000 |
| | H₃BO₃ | 6.200 | 3.000 |
| | Na₂MoO₄2H₂O | 0.025 | 0.025 |
| | NH₂PO₄ | 85.000 | 340.000 |
| | NaFeEDTA | 36.700 | 28.000 |
| growth | | 10 IAA, 0.5 BA | 1.0 IAA |
| regulators | | | |
| carbon | sucrose | 7% | 7% |
| sources | | | |
| PH | | 5.8 | 5.8 |

Based on the results obtained in suspension culture, growth medium (GM) and production medium (PM) were formulated for two stage cultures. 25 days after culture in growth medium, the growth medium was replaced with fresh production medium. Cells were harvested by suction filtration after 10-day incubation period in the test medium.

When the optimal condition for cell growth is different from that for synthesis of secondary metabolites, two stages culture system can improve productivity by optimizing conditions separately for cell growth and product formation. Growth medium and production medium were formulated based on the previous results for optimal nutrient constituents. It was noteworthy that several conditions such as changing concentrations of nitrate, phosphate, carbon source and growth regulators. Thus, we attempted to find conditions for two stage cultures to ensure both maximum growth in GM (growth medium) and maximum production in PM (production medium). After 10 days of two stages culture, the berberine content in cork tree cells was increased to 5.06 mg/g dry weight (Table 7). This result showed the optimization of chemical condition and establishment of two stages culture can enhance the product yield of berberine.

TABLE 7
Comparison between one stage culture and two
stages culture on cell growth

| | cell growth D.W. | berberine content |
|---|---|---|
| cultures | (mg/flask) | (mg/g dry wt) |
| first stage | 25030 | 2.350359 |
| cultures | | |
| (GM -> GM) | | |
| second stage | 29045 | 5.065698 |
| sultures | | |
| (GM -> PM) | | |

### INDUSTRIAL APPLICABILITY

The present invention provides a composition for preventing plant pathogens containing extract of Coptis chinensis Franch., Phellodendron genus plant, Sanguinaria Canadensis or Chelidonium majus var. asiaticum by organic solvent and/or water or purified berberine or derivatives thereof and a method preparing the same. The exptracts of Coptis rhizome, Phellodendron genus plant, Sanguinaria canadensis or Chelidonium majus var. asiaticum or berberine or derivatives thereof may be used for preventing various plant pathogens such as plant powdery mildew, anthracnose of pepper, damping-off of cucumber, gray mold rot of strawberry. The establishment of cell culture such as two stages culture system can enhance the product yield of secondary metabolotes of plants such as berberine.

Those skilled in the art will appreciate that the conceptions and specific embodiments disclosed in the foregoing description may be readily utilized as a basis for modifying or designing other embodiments for carrying out the same purpose of the present invention. Those skilled in the art will also appreciate that such equivalent embodiments do not depart from the spirit and scope of the invention as set forth in the appended claims.

What is claimed is:

1. A composition for preventing at least one plant disease resulted from infection of a plant pathogen containing extract of at least one plant selected from the group consisting of Coptis rhizoma, Phellodendron genus plant, Sanguinaria canadensis and Chelidonium majus var. asiaticum, berberine purified therefrom or derivatives thereof, or synthesized berberine or derivative thereof.

2. The composition according to claim 1, wherein the Phellodendron genus plant is Phellodendron amurense or Phellodendron insulare.

3. The composition according to claim 1, is the berberine or derivatives thereof has the following chemical formula: 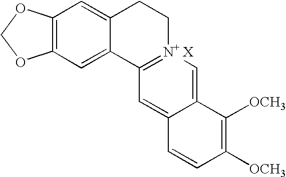
wherein X is Cl⁻, I⁻ or Br⁻.

4. The composition according to claim 1, wherein the plant disease is plant powdery mildew, anthracnose of pepper, damping-off of cucumber or gray mold rot of strawberry.

5. The composition according to claim 1, wherein the plant pathogen is Colletrotrichum gloeosporiodes, Pythium ultimum or Botrytis cinerea.

6. The composition according to claim 1, wherein the extract or the berberine purified therefrom is included at concentration of 1 to 1000 μg/ml.

7. The composition according to claim 1, wherein the extract or the berberine purified therefrom is included at concentration of 10 to 1000 μg/ml.

8. A method for preparing the composition of claim 1 comprising the following steps:
1) extracting at least one plant selected from the group consisting of Coptis Rhizoma (Coptis chinensis Franch.), Phellodendron genus plant, Sanguinaria canadensis and Chelidonium majus var. asiaticum by water, methanol or ethanol or combination thereof after cutting;
2) sedimenting and filtering of extract of step 1; and
3) concentrating extracta of step 2).

9. The method according to claim 8, wherein the Phellodendron genus plant is Phellodendron amurense or Phellodendron insulare.

10. A method for preparing berberine or derivatives thereof comprising the flowing steps:
1) culturing a callus of Phellodendron genus plant using a growth medium;
2) culturing the callus of step 1 using a production medium; and
3) purifying berberine or derivatives thereof from the cultured callus of step 2.

11. The method according to claim 10, wherein the Phellodendron genus plant is Phellodendron amurense or Phellodendron insulare.

12. The method according to claim 10, wherein the growth medium comprises 0.048˜0.19% of KNO₃, 0.009˜0.036% of MgSO₄H₂O, 0.00085˜0.0034% of MnSO₄7H₂O, 0.00043˜0.0017% of ZnSO₄7H₂O, 0.00000013˜0.000005% of CuSO₄5H₂O, 0.017˜0.066% of CaCl₂2H₂O, 0.000042˜0.00017% of KCl, 0.00000013˜0.000005% of CoCl₂2H₂O, 0.083˜0.33% of NH₄NO₃, 0.00031˜0.0012% of H₂BO₃, 0.00000013˜0.000005% of Na₂MoO₄2H₂O, 0.0043˜0.017% of NH₂PO₄, 0.0018˜0.0073% of NaFeEDTA, 0.0005˜0.002% of indole-3-acetic acid (IAA), 0.000025˜0.0001% of benzylamino purine (BA) and 5˜9% of sucrose.

13. The method according to claim 10, wherein the production medium comprises 0.095˜0.38% of KNO₃, 0.0125˜0.05% of MgSO₄H₂O, 0.00085˜0.0034% of MnSO₄7H₂O, 0.00043˜0.0017% of ZnSO₄7H₂O, 0.00000013˜0.000005% of CuSO₄5H₂O, 0.017˜0.066% of CaCl₂2H₂O, 0.000042˜0.00017% of KCl, 0.00000013˜0.000005% of CoCl₂2H₂O, 0.083˜0.33% of NH₄NO₃, 0.00015˜0.00060% of H₂BO₃, 0.00000013˜0.000005% of Na₂MoO₄2H₂O, 0.017˜0.068% of NH₂PO₄, 0.0014˜0.0056% of NaFeEDTA, 0.0005˜0.002% of indole-3-acetic acid (IAA), 0.000025˜0.0001% of benzylamino purine (BA) and 5˜9 of sucrose.

14. A method for preventing at least one plant disease resulted from infection a plant pathogen comprising treating the composition of claim 1 to plants.

15. The method according to claim 14, wherein the plant disease is plant powdery mildew, anthracnose of pepper, damping-off of cucumber or gray mold rot of strawberry.

16. The method according to claim 14, wherein the plant pathogen is Colletrotrichum gloeosporiodes, Pythium ultimum or Botrytis cinerea.
